(19) United States
(12) Patent Application Publication
(10) Pub. No.: US 2024/0196949 A1
(43) Pub. Date: Jun. 20, 2024

(54) INFANT FORMULA

(71) Applicant: John KAPELERIS, Queensland (AU)

(72) Inventors: John KAPELERIS, Queensland (AU); Guillermo TOKMAN, Queensland (AU)

(21) Appl. No.: 18/285,806

(22) PCT Filed: Apr. 6, 2022
(86) PCT No.: PCT/AU2022/050305

§ 371 Date: Oct. 5, 2023

(30) Foreign Application Priority Data
Apr. 6, 2021 (AU) 2021900996

(51) Int. Cl. A23L 33/00; A61K 38/40; A61K 38/47; A61K 39/395; A61K 45/06

(52) U.S. Cl. CPC A23L 33/40; A61K 38/40; A61K 38/47; A61K 39/39516; A61K 45/06; C12Y 302/01017

(57) ABSTRACT

An infant formula composition that comprises particular immunologically active constituents of human breast milk, namely lysozyme and/or at least one immunoglobulin such as immunoglobulin A and or Immunoglobulin G. The infant formula further comprises lactoferrin. The concentration of lysozyme is about 0.1 to 1.0 wt % of the composition, the concentration of the immunoglobulin is about 0.1 to 1.5 wt % of the composition and the concentration of lactoferrin is about 0.1 to 2.0 wt %. The infant formula further comprises one or more vitamins, fats, proteins, carbohydrates, elements such as one or more metals, halogens, phosphorus and selenium, one or more quaternary ammonium compounds such as choline and/or carnitine, one or more amino acids such as taurine, and/or prebiotics and/or probiotics.

### TECHNICAL FIELD

THIS INVENTION relates to an infant formula composition. More particular, this invention relates to an infant formula composition that comprises particular immunologically active constituents of human breast milk.

### BACKGROUND

Infant formulas are typically used as replacements of, or supplements to, human breast milk. Generally, infant formulas include proteins, fats, carbohydrates,
vitamins, minerals and other molecular components that are at least similar to those found in human breast milk and at concentrations that provide suitable nutrition to the infant.

Infant formulas may be particularly useful where a mother cannot breast
feed an infant due to the infant not “latching” to the breast and/or lacking a strong
sucking reflex or due to breast ailments such as mastitis or cracked nipples. Supplementary use may be required where breast feeding is infrequent or intermittent, such as for working mothers, or where the mother is ill or malnourished, which can adversely affect the quantity and quality of breast milk. Even in the absence of any particular breast-feeding problems, infant formulas may
simply provide a convenient and nutritious alternative to breast feeding.

Infant formulas may be manufactured in the form of dried, granular or powdered compositions that can be reconstituted with water, or may be in the form of liquid “ready to go” formulations.

### SUMMARY

The present invention is broadly directed to an infant formula that includes immunologically active components of human breast milk that are not typically present in prior art infant formulas. In a particular broad form, the infant formula comprises lysozyme and/or at least one immunoglobulin such as immunoglobulin A
and/or immunoglobulin G. Preferably, the infant formula further comprises lactoferrin.

In one aspect, the invention provides a composition for use as an infant formula, said composition comprising lysozyme and/or an immunoglobulin.

In an embodiment, the composition comprises lysozyme and/or at least one immunoglobulin selected from IgA and IgG.

In an embodiment, the concentration of lysozyme is about 0.1 to 1.0 wt % of the composition, preferably about 0.2 to 0.8 wt % of the composition, more preferably about 0.4 to 0.7 wt % of the composition or even more preferably about
0.52 wt % of the composition.

In an embodiment, the concentration of immunoglobulin is about 0.1 to 1.5
wt % of the composition, preferably about 0.3 to 1.2 wt % of the composition, more preferably about 0.6 to 1.0 wt % of the composition or even more preferably about
0.85 wt % of the composition.

In a particular embodiment, the composition further comprises lactoferrin. In an embodiment, the concentration of lactoferrin is about 0.1 to 2.0 wt % of
the composition.

Suitably, the composition further comprises one or more vitamins, fats, proteins, carbohydrates, elements such as one or more metals, halogens, phosphorus and selenium, one or more quaternary ammonium compounds such as choline and/or carnitine, one or more amino acids such as taurine and/or prebiotics.

In an embodiment, the composition is substantially as set forth in Table 1.

Suitably, the composition is in a dried, powered or granular form that can be reconstituted with water to produce an infant formula suitable for feeding to an infant.

In another aspect, the invention provides an infant formula comprising
lysozyme and/or an immunoglobulin.

Suitably, the infant formula is in a liquid form suitable for feeding to an infant.

In an embodiment, the infant formula comprises lysozyme and/or at least one immunoglobulin selected from IgA and IgG.

In an embodiment, the concentration of lysozyme is about 0.01 to 0.15 30 g/100 mL, 0.03 to 0.12 g/100 mL, about 0.05 to 0.10 g/100 mL of the infant formula or preferably about 0.07 g/100 mL of the infant formula.

In an embodiment, the concentration of immunoglobulin is about 0.01 to 0.25 g100 mL, about 0.02 to 18 g/100 mL, about 0.04 to 15 g/100 mL or preferably about
0.11 g/100 mL of the infant formula.

In a particular embodiment, the infant formula further comprises lactoferrin.

In an embodiment, the concentration of lactoferrin is about 0.03 to 0.30 g/100 mL or preferably about 0.18 g/100 mL of the infant formula.

Suitably, the infant formula further comprises one or more vitamins, fats, proteins, carbohydrates, elements such as one or more metals, halogens, phosphorus and selenium, one or more quaternary ammonium compounds such as
choline and/or carnitine, one or more amino acids such as taurine and/or prebiotics. In an embodiment, the infant formula is substantially as set forth in Table 1.

In a further aspect, the invention provides a method of producing an infant formula, said method including the step of combining the composition of the first-mentioned aspect with water to thereby produce the infant formula.

This aspect also provides an infant formula produced by the method of this aspect.

In another further aspect, the invention provides a method of feeding an infant, said method including the step of administering the infant formula of the second-mentioned aspect or the infant formula produced by the method of the third
mentioned aspect to an infant.

### DETAILED DESCRIPTION

The present invention is at least partly predicated on the use of lysozyme and immunoglobulins such as IgA in an infant formula to thereby provide an infant
formula with human breast milk components that improve the nutritional and immunological quality of the infant formula. Preferably, the infant formula further comprises lactoferrin. This provides a nutritious, safe and easy to use composition and infant formula that provides additional human proteins such as lysozyme and immunoglobulins to more closely resemble or approximate human breast milk
and/or provide enhanced immunological activity.

Throughout this specification, unless otherwise indicated, “comprise”, “comprises” and “comprising” are used inclusively rather than exclusively, so that a
stated integer or group of integers may include one or more other non-stated integers or groups of integers.

It will also be appreciated that the indefinite articles “a” and “an” are not to be read as singular indefinite articles or as otherwise excluding more than one or
more than a single subject to which the indefinite article refers. For example, “a” protein includes one protein, one or more proteins or a plurality of proteins.

The term “about” is used herein to refer to a tolerance or variation in a stated amount. The tolerance or variation may be no more than ±10%, ±9%, ±8%, ±7%, ±6%, ±5%, ±4%, ±3%, ±2% or ±1% of a stated amount.

As used herein, the term “protein” refers to an amino acid polymer which may comprise natural or non-natural amino acids, D- or L-amino acids. Proteins may be in native or recombinant form. Proteins may further comprise other moieties such as carbohydrates (e.g. glycoproteins), lipids (e.g. lipoproteins, glycolipoproteins) and/or nucleic acids (e.g. ribonucleoproteins).

As used herein, the term “fat” includes and encompasses lipids, waxes, fatty acids, glycerides and sterols inclusive of salts thereof, although without limitation thereto. Typically, fats comprise one or more hydrocarbon (i.e aliphatic) chains and/or rings that may be saturated or mono-, di- or poly-unsaturated. Unsaturated fatty acid chains may be in cis or trans form. Typically, fatty acid aliphatic chains
comprise about 5-22 carbon atoms.

As used herein, the term “carbohydrate” refers to molecules that comprise carbon, oxygen and hydrogen atoms, including but not limited to saccharides (e.g. monosaccharides, oligosaccharides, polysaccharides) such as sugars, starches and cellulose.

In one aspect, the invention provides a composition for use as an infant formula, said composition comprising lysozyme and/or an immunoglobulin.

“Lysozyme” is a protein also known as muramidase or N-acetylmuramide glycanhydrolase, and functions as an antimicrobial enzyme that forms part of the innate immune system. Lysozyme is a glycoside hydrolase that catalyzes
the hydrolysis of 1,4-beta-linkages between N-acetylmuramic acid and N-acetyl-D-glucosamine residues in peptidoglycan, which is the major component of gram-positive bacterial cell wall. This hydrolysis in turn compromises the integrity of
bacterial cell walls causing lysis of the bacteria. Lysozyme is normally present in human breast milk and provides a source of “transferred” innate immunity to the infant.

Although not wishing to be bound by theory, it is proposed that the addition
of lysozyme to the composition and infant formula disclosed herein may at least partly enhance or improve the immunological activity of the infant formula.

In an embodiment, the concentration of lysozyme is about 0.1 to 1.0 wt % of the composition, preferably about 0.2 to 0.8 wt % of the composition, more preferably about 0.4 to 0.7 wt % of the composition or about 0.52 wt % of the
composition.

Lysozyme can be sourced from chicken egg white, cauliflower, bovine milk, honey, human serum or milk, fruits, vegetables, tofu, bean curd, seafoods, meats, potato and/or burdock. Lysozyme may also be produced biosynthetically, such as a recombinant protein.

As hereinbefore described, the composition further comprises an immunoglobulin.

As used herein, an “immunoglobulin” is an antigen-binding protein (typically referred to as an “antibody”), or fragment thereof, that is encoded by a mammalian immunoglobulin gene complex. Typically, immunoglobulins comprise isotypes such
as IgM, IgG, IgA, IgD and IgE. Isotypes may be further characterized into subclasses such as IgG1, IgG2, IgG3 and IgG4, which primarily differ in their constant region, particularly in their hinge and upper CH2 domains. The immunoglobulin may be polyclonal or monoclonal in native or recombinant form and/or may comprise antibody fragments such as scFv, Fc, Fab and/or F(ab′)2 fragments. Synthetic
antibodies are also contemplated such as diabodies, triabodies and/or other polyspecific synthetic antibodies.

Immunoglobulins, particularly IgA, is normally present in human breast milk and thereby provides maternally-derived anti-microbial antibody protection to the infant.

Although not wishing to be bound by theory, it is proposed that the addition of immunoglobulins such as IgA and/or IgG to the composition and infant formula
disclosed herein may at least partly enhance or improve the immunological activity of the infant formula.

In an embodiment, the concentration of immunoglobulin is about 0.1 to 1.5 wt % of the composition, preferably about 0.3 to 1.2 wt % of the composition, more
preferably about 0.6 to 1.0 wt % of the composition or about 0.85 wt % of the composition.

Suitably, the composition comprises an immunoglobulin selected from IgA and IgG.

IgG may be sourced naturally, such from IgG present in bovine milk. IgA may
be sourced from either commercial sources or may be produced biosynthetically, such as a recombinant protein. Preferably, IgA is human IgA.

In a particular embodiment, the composition further comprises lactoferrin. Lactoferrin is an ˜80 kD globular glycoprotein transferrin (“lactotransferrin”)
that binds and transfers iron to cells and controls the level of free iron in the blood
and in external secretions. It is present in the milk of humans and other mammals, in the blood plasma and in neutrophils and is one of the major proteins of virtually all exocrine secretions of mammals, such as saliva, bile, tears and pancreatic secretions. Lactoferrin also has antimicrobial activity (e.g against bacteria and fungi) and is part of the innate immune system, particularly at mucosal sites.

In an embodiment, the concentration of lactoferrin is about 0.1 to 2.0 wt % of the composition.

Preferably, lactoferrin may be of bovine or human origin and may be obtained from commercial sources such as Freedom Foods Group Limited.

Typically, the composition may further comprise other proteins such as
albumins, including α-lactalbumin and serum albumin, although without limitation thereto.

Suitably, the composition further comprises one or more vitamins, fats, carbohydrates, elements such as metals, halogens, phosphorus and/or selenium, quaternary ammonium compounds, amino acids and/or prebiotics, or probiotics.

Vitamins may comprise B complex vitamins such as vitamin B6, vitamin B12, niacin, thiamin, folate, biotin, riboflavin and/or pantothenic acid, vitamin C, vitamin D, vitamin K and/or vitamin E.

Fats may include fatty acids such as linolenic acid, alpha-linolenic acid, arachidonic acid, docosahexaenoic acid (DHA), omega 3 and/or omega 6 fatty acids. The composition may further comprise one or more elements such as metals.

Metals may be in ionic or atomic metal form. Metals may include transition metals,
alkali metals and/or alkaline earth metals. Non-limiting examples of metals include sodium, calcium, chromium, boron, potassium, iron, copper, manganese, magnesium, molybdenum and/or zinc.

The composition may further comprise one or more elements such as selenium and phosphorus and/or halogens such as chlorine (e.g. as chloride ion)
and/or iodine, although without limitation thereto.

The composition may further comprise one or more quaternary ammonium compounds such as choline and/or carnitine. The composition may further comprise one or more amino acids such as taurine.

The composition may further comprise carbohydrates such as inositol,
galacto-oligosaccharide (GOS) prebiotic and fructo-oligosaccharide (FOS) prebiotic, although without limitation thereto, and may include probiotics.

A particular embodiment of the composition is set forth in Table 1, which includes particular concentrations and tolerances of each of the constituents of the composition.

As will be appreciated from the foregoing, the composition disclosed herein
is suitable for use as an infant formula. In one embodiment, the composition is provided in a dried or dehydrated powdered or granular form. Methods of manufacturing dried or dehydrated powdered or granular infant formula compositions are well known in the art and typically include combining the
components, such as disclosed herein with particular reference to Table 1, into a
water-based slurry and then drying or dehydrating the slurry to produce the powdered or granular infant formula composition. This process also typically includes pasteurization and homogenization steps prior to drying or dehydrating the slurry. A particularly important consideration of the manufacturing process is that
the composition and formula are safe to administer to the infant.

Suitably, the dried or dehydrated powdered or granular composition is combined with water to produce the infant formula.

Preferably, the composition is combined with water at a ratio of about 13 grams composition to 100 mL water. However, it will be appreciated that this ratio may readily be varied according to the nutritional needs of the infant.

In an alternative embodiment, the infant formula is provided as a pre
prepared, liquid infant formula which does not require reconstitution with water.

In one embodiment of the infant formula, the concentration of lysozyme is about 0.01 to 0.15 g/100 mL, 0.03 to 0.12 g/100 mL, about 0.05 to 0.10 g/100 mL of the infant formula or preferably about 0.07 g/100 mL of the infant formula.

In one embodiment, the concentration of immunoglobulin, such as IgA or IgG,
is about 0.01 to 0.25 g/100 mL, about 0.02 to 0.18 g/100 mL, about 0.04 to 0.15 g/100 mL or preferably about 0.11 g/100 mL of the infant formula.

The infant formula may further comprise lactoferrin.

In one embodiment, the concentration of lactoferrin is about 0.03 to 0.30 g/100 mL, or preferably about 0.18 g/100 mL of the infant formula.

Typically, the infant formula may further comprises other proteins such as albumins, including α-lactalbumin and serum albumin, although without limitation thereto.

Suitably, the infant formula further comprises one or more vitamins, fats, carbohydrates, one or more elements such as metals, halogens, phosphorus and/or
selenium, one or more quaternary ammonium compounds, one or more amino acids and/or one or more prebiotics, and/or probiotics.

Vitamins may comprise B complex vitamins such as vitamin B6, vitamin B12, niacin, thiamin, folate, biotin, riboflavin and/or pantothenic acid, vitamin C, vitamin D, vitamin K and/or vitamin E, although without limitation thereto.

Fats may include fatty acids such as linolenic acid, alpha-linolenic acid, arachidonic acid, docosahexaenoic acid (DHA), omega 3 and/or omega 6 fatty acids. Metals may be in ionic or atomic metal form. Metals may include transition metals, alkali metals and/or alkaline earth metals. Non-limiting examples of metals include sodium, chromium, boron, calcium, potassium, iron, copper, manganese,
magnesium, molybdenum and/or zinc.

The infant formula may further comprise one or more elements such as selenium and phosphorus and/or halogens such as chlorine (e.g. as chloride ion) and/or iodine, although without limitation thereto.

The infant formula may further comprise one or more quaternary ammonium
compounds such as choline and/or carnitine. The infant formula may further comprise one or more amino acids such as taurine.

The infant formula may further comprise carbohydrates such as inositol, galacto-oligosaccharide (GOS) prebiotic and fructo-oligosaccharide (FOS) prebiotic, although without limitation thereto, and may include probiotics.

A particular embodiment of the infant formula is set forth in Table 1, which includes particular concentrations of each of the constituents of the infant formula.

In another further aspect, the invention also provides a method of feeding an infant, said method including the step of administering the infant formula of the second-mentioned aspect or the infant formula produced by the method of the third
mentioned aspect to an infant.

Typically, the infant will be a newborn baby or up to about three (3) years of age, although it is preferred that the infant is no older than about 12-18 months old. Thus, while the infant formula is produced ideally for an infant, it may also be suitable for administration to a young child or “toddler”.

The amount or volume of infant formula administered to the infant or young child depend, at least in part, on the age of the infant or young child and whether the infant formula is administered as a “supplement” to other nutrition (as is typically the case with a young child) or is essentially the sole source of nutrition. By way of example only, from five days to three months, a full-term, healthy baby will need
about 150 mL of prepared formula per kilogram (kg) of body weight, every day. For example, a baby who weighs 3 kg will need 450 mL of prepared infant formula each day.

Typically, the formula is administered to the infant or young child by way of a bottle that comprises a teat, whereby the sucking action of the infant or young child
assists ingestion of the infant formula.

In some circumstances where the infant has difficulty sucking, gastrointestinal defects, respiratory problems or other health problems, the infant
formula may be suitable for administration to the infant by way of a feeding tube inserted directly into the stomach via the mouth.

It will be appreciated from the foregoing that the invention provides a nutritious, safe and easy to use composition and infant formula that provides
additional human proteins such as lysozyme and immunoglobulins to more closely resemble or approximate human breast milk and also at least partly enhance the immunological activity of the infant formula.

It will therefore be appreciated that the infant formula and methods disclosed herein are particularly suitable for human infants, however the invention may also
be applied to feeding other mammals, particularly primate infants that may have nutritional needs similar to those of humans.

TABLE 1

| | | Concen- | Concen- | Concen- |
|---|---|---|---|---|
| | | tration | tration | tration |
| | | (amount/100 | (amount/100 | (amount/100 |
| | Unit | gm powder) | mL formula) | Kcal) |
| Energy | kcal | 518 | 67.33 | 100 |
| Protein | g | 10.8 | 1.40 | 2.085 |
| Fat | g | 27 | 3.51 | 5.212 |
| Alpha-linolenic acid | mg | 850 | 110.49 | 164.093 |
| Linolenic acid | mg | 5765 | 749.37 | 1112.934 |
| Carbohydrate | g | 58 | 7.54 | 11.197 |
| Vitamin A | μg | 500.00 | 64.99 | 96.525 |
| | RE | | | |
| Vitamin B6 | μg | 480 | 62.39 | 92.664 |
| Vitamin B12 | μg | 3.5 | 0.45 | 0.676 |
| Vitamin C | μg | 75 | 9.75 | 14.479 |
| Vitamin D | μg | 7.65 | 0.99 | 1.477 |
| Vitamin E | mg α- | 11.5 | 1.49 | 2.220 |
| | TE | | | |
| Vitamin K | μg | 42 | 5.46 | 8.108 |
| Biotin | μg | 17 | 2.21 | 3.282 |
| Niacin | μg | 4000 | 519.94 | 772.201 |
| Folic acid | μg | 70 | 9.10 | 13.514 |
| Pantothenic acid | μg | 4800 | 623.93 | 926.641 |
| Riboflavin | μg | 1500 | 194.98 | 289.575 |
| Thiamin | μg | 750 | 97.49 | 144.788 |
| Calcium | mg | 325 | 42.25 | 62.741 |
| Copper | μg | 454.5 | 59.08 | 87.741 |
| Iodine | μg | 120 | 15.60 | 23.166 |
| Iron | mg | 6.2 | 0.81 | 1.197 |
| Magnesium | mg | 46 | 5.98 | 8.880 |
| Manganese | μg | 104 | 13.52 | 20.077 |
| Phosphorus | mg | 192 | 24.96 | 37.066 |
| Selenium | μg | 16 | 2.08 | 3.089 |
| Zinc | mg | 6 | 0.78 | 1.158 |
| Chloride | mg | 370 | 48.09 | 71.429 |
| Potassium | mg | 510 | 66.29 | 98.456 |
| Sodium | mg | 155 | 20.15 | 29.923 |
| Choline | mg | 53.5 | 6.95 | 10.328 |
| Inositol | mg | 60 | 7.80 | 11.583 |
| Carnitine | mg | 8 | 1.04 | 1.544 |
| Lactoferrin | g | 1.37 | 0.18 | 0.264 |
| Lysozyme | g | 0.52 | 0.07 | 0.100 |
| Immunoglobulin | g | 0.85 | 0.11 | 0.164 |
| DHA (Omega 3) | mg | 13.8 | 1.79 | 2.667 |
| Arachidonic acid | mg | 19.8 | 2.57 | 3.828 |
| (Omega 6) | | | | |
| Prebiotics (GOS) | g | 1.582 | 0.21 | 0.305 |
| Prebiotics (FOS) | g | 0.603 | 0.08 | 0.116 |
| Total Nucleotide 5′ | mg | 12.5 | 1.62 | 2.413 |
| Taurine | mg | 35 | 4.55 | 6.757 |
| L-Carnosine | mg | 212.7 | 27.65 | 41.000 |
| Betaine | mg | 120 | 15.62 | 24.000 |
| Q10 | mg | 388.6 | 50.51 | 75.000 |
| Q8 | mg | 388.6 | 50.51 | 75.000 |

Throughout the specification the aim has been to describe the preferred embodiments of the invention without limiting the invention to any one embodiment or specific collection of features. It will therefore be appreciated by those of skill in the art that, in light of the instant disclosure, various modifications and changes can
be made in the particular embodiments exemplified without departing from the scope of the present invention.

All computer programs, algorithms, patent and scientific literature referred to herein is incorporated herein by reference.

1. A composition for use as an infant formula, said composition comprising lysozyme and/or an immunoglobulin.
2. The composition of claim 1, which comprises lysozyme and/or at least one immunoglobulin selected from IgA and IgG.

2. The composition of claim 1, which comprises lysozyme and at least one immunoglobulin selected from IgA and IgG.

3. The composition of any preceding claim, wherein the concentration of lysozyme is about 0.1 to 1.0 wt % of the composition, preferably about 0.2 to
0.8 wt % of the composition, more preferably about 0.4 to 0.7 wt % of the composition or about 0.52 wt % of the composition.

4. The composition of any preceding claim, wherein the concentration of immunoglobulin is about 0.1 to 1.5 wt % of the composition, preferably about
0.3 to 1.2 wt % of the composition, more preferably about 0.6 to 1.0 wt % of the composition or about 0.85 wt % of the composition.

5. The composition of any preceding claim, wherein the composition further comprises lactoferrin.

6. The composition of claim 6, wherein the concentration of lactoferrin is about 0.1 to 2.0 wt % or preferably about 1.37 wt % of the composition.

7. The composition of any preceding claim further comprising one or more vitamins, fats, proteins, carbohydrates, elements such as one or more metals, halogens, phosphorus and selenium, one or more quaternary ammonium compounds such as choline and/or carnitine, one or more amino acids such as
taurine and/or prebiotics and/or probiotics.

8. The composition of any preceding claim is substantially as set forth in Table 1.

9. The composition of any preceding claim is in a dried, powered or granular form that can be reconstituted with water to produce an infant
formula.

10. An infant formula comprising lysozyme and/or an immunoglobulin.

11. The infant formula of claim 11 which comprises lysozyme and/or at least one immunoglobulin selected from IgA and IgG.

12. The infant formula of claim 12 which comprises lysozyme and at least one immunoglobulin selected from IgA and IgG.
14. The infant formula of any one of claims 11-13, wherein the concentration of lysozyme is about 0.01 to 0.15 g/100 mL, 0.03 to 0.12 g/100 mL, about 0.05 to 0.10 g/100 mL or preferably about 0.07 g/100 mL of the infant formula.

13. The infant formula of any one of claims 11-14, wherein the concentration of immunoglobulin is about 0.01 to 0.25 g/100 mL, about 0.02 to 18 g/100 mL, about 0.04 to 15 g/100 mL or preferably about 0.11 g/100 mL of the infant formula.

14. The infant formula of any one of claims 11-15, wherein the infant formula further comprises lactoferrin.
17. The infant formula of claim 16, wherein concentration of lactoferrin is about 0.03 to 0.30 g/100 mL or preferably about 0.18 g/100 mL of the infant formula.

15. The infant formula of any one of claims 11-17 which further comprises one or more vitamins, fats, proteins, carbohydrates, elements such as one or
more metals, halogens, phosphorus and selenium, one or more quaternary ammonium compounds such as choline and/or carnitine, one or more amino acids such as taurine and/or one or more prebiotics and/or probiotics.

16. The infant formula of any one of claims 11-18, substantially as set forth in Table 1.
20. A method of producing an infant formula, said method including the step of combining the composition of any one of claims 1-10 with water to thereby produce the infant formula.

17. An infant formula produced by the method of claim 20.

18. The infant formula of claim 21, which is the infant formula of any one of claims 11-19.

19. A method of feeding an infant, said method including the step of administering the infant formula of any one of claims 11-19, 21 or 22 to an infant.
